(19) United States
(12) Patent Application Publication
(10) Pub. No.: US 2023/0015045 A1
(43) Pub. Date: Jan. 19, 2023

(54) MEDICAL PRODUCTS INCLUDING SILVER ANTIMICROBIAL, HYDRATION MEIDUM AND A STABILIZING AGENT

(71) Applicant: Hollister Incorporated, Libertyville (US)

(72) Inventors: Tadas S. Sileika, Lake Forest, IL (US); Nicole L. Dean, Antioch, IL (US); Jacqueline MJ Pettigrew, Lindenhurst, IL (US)

(21) Appl. No.: 17/786,008

(22) PCT Filed: Dec. 16, 2020
(86) PCT No.: PCT/US2020/065403

§ 371 Date: Jun. 16, 2022

Related U.S. Application Data
(60) Provisional application No. 62/951,740, filed on Dec. 20, 2019.

(51) Int. Cl. A61L 12/08; A61L 29/08; A61L 29/14; A61L 29/16

(52) U.S. Cl. CPC A61L 12/088; A61L 29/085; A61L 29/143; A61L 29/16; A61L 2300/404; A61L 2300/606; A61M 2025/0056

(57) ABSTRACT

Medical products including hydrophilic coating containing silver antimicrobial, hydration medium and a stabilizing agent.

The present application claims the benefit of and priority to U.S. Provisional Patent Application No. 62/951,740, filed Dec. 20, 2019, which is hereby incorporated herein by reference.

### DESCRIPTION

### Technical Field

The present disclosure generally relates to packaged medical products that contain a medical device having a silver antimicrobial, a hydration medium and a stabilizing agent, such as a masking agent, wherein the stabilizing agent reduces discoloration of the hydration medium caused by silver.

### Background

Some medical devices are required to be hydrated with a hydration medium or stored in a hydration medium, such as liquid water or saline. Such devices may include hydrophilic urinary catheters, contact lenses, etc. These medical devices may be packaged in a package containing the medical device and the hydration medium. In some instances, it may be desirable for the medical device to include a silver antimicrobial. One challenge with including a silver antimicrobial is when the device is packaged in contact with the hydration medium is that the silver or other components of the antimicrobial may cause the hydration medium to become discolored. For example, a clear hydration medium may become reddish from the silver antimicrobial.

When the package is opened, there is a risk that the hydration medium unintentionally spills or leaks from the package. If the discolored hydration medium contacts the user's clothing, the hydration medium may leave a stain on the fabric of the clothing. Furthermore, the user may find a reddish hydration medium unsightly.

Therefore, there remains a need for packaged medical devices that include antimicrobials, a hydration medium and reduces the risk of the hydration medium staining the user's clothes.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In a first aspect, a medical product that includes a package having a cavity. The cavity containing a medical device at least partially made from a hydrophilic polymer and a silver antimicrobial within the hydrophilic polymer. The cavity also containing a hydration medium including a liquid, and a stabilization agent that interacts with one or more components of the silver antimicrobial to reduce discoloration of the hydration medium from the silver antimicrobial.

In another aspect, a urinary catheter product that includes a urinary catheter including a catheter tube and a lubricous coating disposed on the catheter tube, the lubricous coating comprising a hydrophilic polymer. A silver antimicrobial is within the coating. The product also includes a hydration medium including a liquid. The product further includes a stabilization agent that interacts with one or more components of the silver antimicrobial to reduce discoloration of the hydration medium from the silver antimicrobial.

In yet another aspect, a medical device product includes a medical device, a silver antimicrobial, a hydration medium, and a stabilizing agent.

### BRIEF DESCRIPTION OF FIGURES

FIG. 1 is a plan view of a medical device product in accordance with the present disclosure; and

FIG. 2 is cross-sectional view of the medical device of FIG. 1.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

The present disclosure discloses medical device products that include a silver antimicrobial, a hydration medium, and a stabilizing agent, such as a masking agent. The stabilizing agent may be associated with the medical device or the hydration medium. The medical device may be any medical device that utilizes a silver antimicrobial. The antimicrobial may be on the surface of the device or contained or impregnated in the material of the device. In one alternative, the silver antimicrobial may be in a hydrophilic material of the device. The hydrophilic material, such as hydrophilic coatings, includes the silver antimicrobial therein. The hydrophilic material may be a hydrophilic polymer. The silver antimicrobial may be bonded to the polymer or may be in free form within the polymer matrix. The silver antimicrobial may be at the surface of a polymer coating or may be impregnated within or blended with the polymer coating. Furthermore, the silver antimicrobial may be uniform throughout the coating or the coating may have selected portions that have more silver antimicrobial than others.

The medical products also include a hydration medium. The hydration medium may be any suitable hydration medium for hydrating and/or storing the medical device. The hydration medium may include a liquid, such as water, and any suitable additives or agents.

The medical product also includes a stabilizing agent, such as a masking agent, that reduces discoloration of the hydration medium which may result from the silver antimicrobial or components thereof. The stabilizing agent may also enhance the antimicrobial effect. The stabilizing agent may be in the hydration fluid or be associated with the medical device.

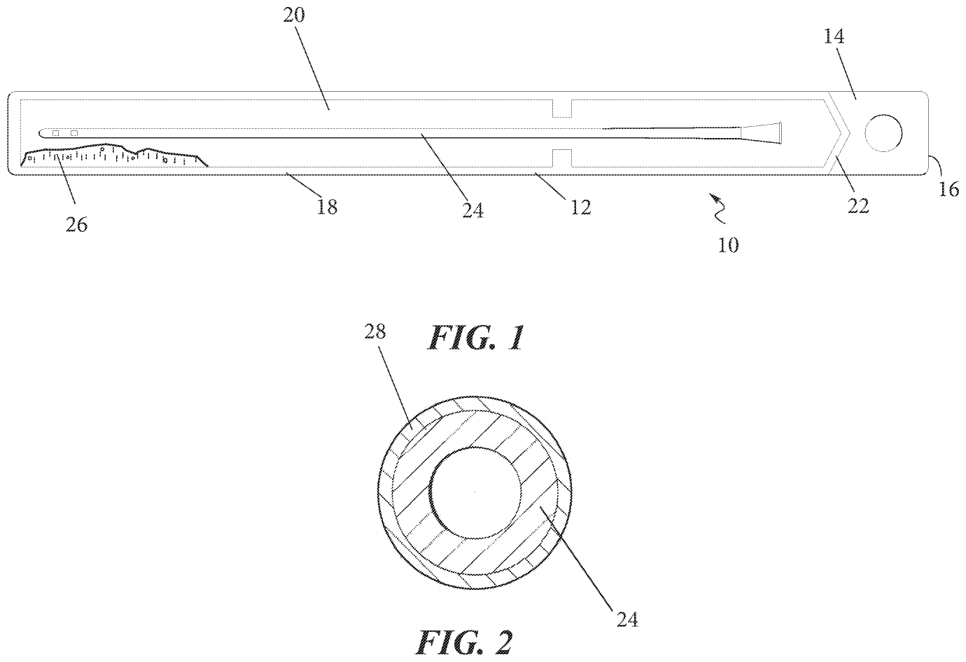

FIG. 1 shows one exemplary medical device product 10. In this figure, the medical device is illustrated as a urinary catheter 24. However, the medical device may be any medical device that is stored in a hydration medium. For example, the medical device may be at least partially made from a hydrophilic material and may be packaged with a hydration medium 26, such as liquid water or saline. The packaged hydrophilic medical device product 10 includes a package 12 that surrounds that catheter 24. In the embodiment illustrated in FIG. 1, the package 12 is formed from a front sheet 14 and back sheet (not shown) that are sealed together to form a peripheral seal 18 and define an internal cavity 20. At the top of the package 12, the front and back sheets may be unattached above top seal 22. The package 12 may be opened by grasping these unattached portions and pulling the front sheet 14 and back sheet away from each other to peal open the package 12 along seal 18. Optionally, the package 12 may be any other suitable package for containing a medical device. For example, the package may be a tear open package. Additionally, the material of the package, optionally, may be made from a gas impermeable material.

A hydrophilic medical device is contained within cavity 20. As mentioned above, in the illustrated embodiment, the hydrophilic medical device is a hydrophilic catheter 24. The hydrophilic catheter 24 may be any suitable hydrophilic catheter that includes a hydrophilic outer surface that becomes lubricous when hydrated with a hydration medium, such as water. For example and referring to FIG. 2, the catheter 24 may include a lubricious hydrophilic coating 28 on the outer surface of the catheter. The hydrophilic coating includes a hydrophilic polymer and a silver antimicrobial within the hydrophilic polymer. The silver antimicrobial may be a silver salt, such as a salt of silver and iodine. In one alternative, the silver antimicrobial may be silver periodate, such as pentasilver hexaoxoiodate (Ag₅IO₆).

A hydration medium, such as a hydration liquid 26, also may be located within the cavity 20. The hydration medium may be loose or free flowing within the cavity 20, or the hydration medium may be in a compartment within the cavity 20 wherein the compartment keeps the hydration medium stored separately from the catheter 24. During manufacturing or just prior to use, the separate compartment may be opened so that the hydration medium 26 is free flowing with the cavity 20 and contacts the catheter. The hydration medium may be water or saline that includes any suitable additives.

The product may also include a stabilizing agent. The stabilizing agent may be in the hydration medium and/or within the hydrophilic coating. The stabilizing agent may be, for example, a salt that includes an anion that binds with silver. For example, the salt may be any suitable alkaline halide salt. Such salts may include a bromine or chlorine anion. The salt may be, for example, magnesium chloride or barium bromide. Furthermore, the stabilizing agent may include a cation that binds with iodine, such as one or more calcium halides. In one embodiment, the stabilizing agent may be calcium chloride. In other embodiments the stabilizing may be calcium phosphate, calcium sulfate, calcium fluoride, calcium bromide. When the stabilizing agent, such as calcium chloride or any of the other suitable salt, is within the hydration medium, the stabilizing agent may be in an amount of 0.0001 wt % to 10 wt % of the hydration medium, preferably about 1 wt %. The stabilizing agent may also serve to increase the antimicrobial performance of the device.

When catheter 24 is stored in contact with hydration medium, silver and/or iodine ions (when silver iodine is used) may migrate from the catheter and into the hydration medium. When the stabilizing agent is calcium chloride, free silver ions bind with the chloride ions from calcium chloride, precipitating as a white solute into the coating. A transient amount of free silver ions remains in solution, without metallization (via reduction) into metallic silver, which otherwise would cause the discoloration. Similarly, when iodide is present, the iodide ions form a salt with the calcium ions from calcium chloride, creating colorless salt. Otherwise, free iodine would cause an amber color to emerge for the solution.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A medical product, comprising:
a package having a cavity containing:
a medical device at least partially made from a hydrophilic polymer and a silver antimicrobial within the hydrophilic polymer;
a hydration medium including a liquid; and
a stabilization agent that interacts with one or more components of the silver antimicrobial to reduce discoloration of the hydration medium from the silver antimicrobial.

2. The product of claim 1, wherein the hydration medium includes the stabilization agent.

3. The product of claim 1, wherein the stabilization agent is within the hydrophilic polymer.

4. The product of claim 1, wherein the medical device comprises a urinary catheter.

5. The product of claim 4, wherein the urinary catheter includes a coating comprises the hydrophilic polymer.

6. The product of claim 1, wherein the silver antimicrobial comprises a silver salt.

7. The product of claim 1, wherein the silver antimicrobial comprises silver and iodine.

8. The product of claim 1, wherein the silver antimicrobial comprises silver periodate.

9. The product of claim 1, wherein the silver antimicrobial comprises pentasilver hexaoxoiodate (Ag₅IO₆).

10. The product of claim 1, wherein the stabilization agent comprises a salt that includes an anion that binds with silver.

11. The product of claim 1, wherein the stabilization agent comprises a salt that includes a cation that binds with iodine.

12. The product of claim 1, wherein the stabilization agent comprises calcium chloride.

13. The product of claim 2, wherein the stabilization agent comprises calcium chloride 0.0001 wt % to 10 wt % of the hydration medium, preferably about 1 wt %.

14. The product of claim 13, wherein the hydration medium includes other additives.

15. A urinary catheter product, comprising:
a urinary catheter including a catheter tube;
a lubricous coating disposed on the catheter tube, the lubricous coating comprising a hydrophilic polymer;
a silver antimicrobial within the coating;
a hydration medium including a liquid; and
a stabilization agent that interacts with one or more components of the silver antimicrobial to reduce discoloration of the hydration medium from the silver antimicrobial.

16. The product of claim 15, wherein the hydration medium includes the stabilization agent.

17. The product of claim 15, wherein the stabilization agent is within the hydrophilic polymer.

18. The product of claim 15, wherein the silver antimicrobial comprises a silver salt.

19. The product of claim 15, wherein the silver antimicrobial comprises silver and iodine.

20. The product of claim 15, wherein the silver antimicrobial comprises silver periodate.

21.-28. (canceled)
